(19) United States
(12) Patent Application Publication
(10) Pub. No.: US 2009/0318695 A1
(43) Pub. Date: Dec. 24, 2009

(54) PROCESSES FOR THE PREPARATION OF VARENICLINE AND INTERMEDIATES THEREOF

(76) Inventors: Vinod Kumar Kansal, Haryana (IN); Suhail Ahmad, New Delhi (IN); Amit Gupta, New Delhi (IN)

Correspondence Address: KENYON & KENYON LLP, ONE BROADWAY, NEW YORK, NY, 10004, US

(21) Appl. No.: 12/487,169

(22) Filed: Jun. 18, 2009

Related U.S. Application Data
(60) Provisional application No. 61/132,635, filed on Jun. 19, 2008.

(51) Int. Cl. C07D 471/08; C07D 221/22

(52) U.S. Cl. 544/343; 546/97

(57) ABSTRACT

The invention provides an improved process for the preparation and purification of Varenicline and intermediates for the preparation of Varenicline.

### RELATED APPLICATIONS

This application claims benefit of U.S. Provisional Patent Application No. 61/132,635, filed Jun. 19, 2008, the contents of which are incorporated herein in its entirety by reference.

### FIELD OF INVENTION

The present invention provides an improved process for the preparation and purification of Varenicline and intermediates in the preparation of Varenicline.

### BACKGROUND OF THE INVENTION

CHANTIX™ tablets contain the active ingredient, Varenicline, as the tartrate salt, which is a partial agonist selective for α₄β₂ nicotinic acetylcholine receptor subtypes.

Varenicline tartrate salt is a powder, which is a white to off-white to slightly yellow solid, with the chemical name: 7,8,9,10-tetrahydro-6,10-methano-6H-pyrazino[2,3-h][3]benzazepine, (2R,3R)-2,3-dihydroxybutanedioate (1:1). Varenicline tartrate is highly soluble in water, has a molecular weight of 361.35 Daltons, and a molecular formula of C₁₃H₁₃N₃.C₄H₆O₆. The chemical structure of Varenicline tartrate is:

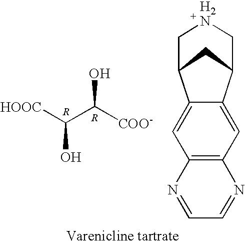

Varenicline is a partial agonist of the α₄β₂ subtype of the nicotinic acetylcholine receptor. In addition it acts on α₃β₄, and acts weakly on α₃β₂ and α₆-containing receptors. A full agonism was displayed on α₇-receptors.

Varenicline is indicated for smoking cessation. It is an alternative to NRTs (nicotinic acetylcholine receptor) and agonist medication, and has demonstrated greater efficacy in comparable studies.

The FDA has approved Varenicline use for twelve weeks. If smoking cessation has been achieved by that time, the use of Varenicline may be continued for another twelve weeks.

U.S. Pat. No. 6,410,550 (US 550′) describes a process for the preparation of Varenicline and the intermediates thereof. US 550′ discloses processes for the synthesis of aryl fused azapolycyclic compounds, obtained in a step by step manner. The disclosed processes are not industrially applicable in terms of reaction times and reagents used. For example, the reaction time disclosed in US 550′ is typically up to 60 hours.

The present invention provides an improved process for obtaining the Varenicline compound in a commercially viable and economical process.

### SUMMARY OF THE INVENTION

The present invention is directed to a process for preparing Varenicline and intermediates of Varenicline The process comprises:

a) Adding a di-halo substituted benzene in the presence of a solvent and a haloalkane to cyclopentadiene to obtain a compound of formula (IV), 1,4-Dihydro-1,4-methano-naphthalene in a Grignard reaction;

b) Treating the compound of formula (IV) obtained in step (a) with a catalyst in the presence of a solvent and, subsequently, an oxidizing agent to obtain a compound of formula (V), 1,2,3,4-Tetrahydro-1,4-methano-naphthalene-2,3 diol;

c) Adding an oxidizing agent, a phase transfer catalyst, a protecting agent, and a reducing agent to the compound of formula (V) obtained in step (b) to obtain a compound of formula (VI), 10-Benzyl-10-aza-tricyclo[6.3.1.0^{2,7}]dodeca-2(7),3,5-triene, and, optionally, purifying the compound of formula VI in a process step, comprising contacting the compound of formula VI with water and an alkali metal dihydrogen phosphate;

d) Adding HCl to the compound of formula VI obtained in step (c) to obtain the compound 10-Benzyl-10-aza-tricyclo[6.3.1.0^{2,7}]dodeca-2(7), 3,5-triene HCl, and, subsequently, debenzylating the 10-Benzyl-10-aza-tricyclo[6.3.1.0^{2,7}]dodeca-2(7), 3,5-triene HCl to obtain a compound of formula (VII), 10-Aza-tricyclo[6.3.1.0^{2,7}]dodeca-2(7)3,5-triene hydrochloride;

e) Adding a solvent and a fluorinating agent to the compound of formula (VII) obtained in step (d) to obtain a compound of formula (VIII), 1-(10-Aza-tricyclo[6.3.10^{2,7}]dodeca-2 (7), 3,5-triene 10-yl)-2,2,2-trifluoro-ethanone;

f) Adding a nitrating source in the presence of a solvent and a Lewis acid to the compound of formula (VIII) obtained in step (e), to obtain a compound of formula (IX), 1-(4,5-dinitro-10-aza-tricyclo[6.3.1.0^{2,7}]dodeca-2(7), 3,5-triene-10-yl)-2,2,2-trifluoroethanone, and, subsequently, reducing the compound of formula (IX) by hydrogenation to obtain a compound of formula (X), 1-(4,5-Diamino-10-aza-tricyclo[6.3.1.0^{2,7}]dodeca-2(7),3,5-triene-10-yl)-2,2,2-trifluoro-ethanone.

g) Cyclising the compound of formula (X) obtained in step (f) with a 40 percent aqueous glyoxal solution to obtain a compound of formula (XI), 1-(5,8,14-Triazatetracyclo[10.3.1.0^{2,11}.0^{4,9}]hexadeca-2(11),3,5,9-pentaene)-2,2,2-trifluoro-ethanone; and purifying the compound of formula XI in a process step, comprising combining or contacting the compound of formula XI with an acid; and

h) Deprotecting the compound of formula XI obtained in step (g) to obtain Varenicline base.

The present invention also provides a process for preparing Varenicline L-tartrate comprising, obtaining Varenicline base according to the process described above, and converting the obtained Varenicline base to Varenicline L-tartrate.

### DETAILED DESCRIPTION

The present invention is directed towards an improved process for the preparation of Varenicline and intermediates of the process. The process of the invention significantly reduces the overall cost of the preparation of Varenicline, and allows the use of non-carcinogenic and environmentally friendly reagents.

The present invention provides significantly reduced process times, making the process of the invention more economical than prior art processes for preparing Varenicline. 1,2-dibromoethane is used as a catalyst to initiate the Grignard reaction of the process, which is safer to use than hazardous and moisture sensitive reagents, such as ethyl magnesium bromide.

The present invention also provides processes for preparing certain intermediates in the process for the preparation of Varenicline having a higher purity. The processes of the present invention for the purification of the intermediates are commercially viable, as the reagents used, e.g., potassium dihydrogen phosphate and hydrochloric acid, are commonly used in industry, and provide for the use of separation methods other than column chromatography, making the process of the invention less costly, as column chromatography is expensive and time consuming on an industrial scale.

As used herein, the term “solvents” refers to organic and inorganic solvents. The reaction of the invention is conducted in a solvent selected from the group consisting of halogenated hydrocarbons, C6 to C14 aromatic hydrocarbons, C1 to C5 alcohols, C2 to C7 esters, C2 to C7 ethers, C1 to C5 carboxylic acids, water, and mixtures thereof.

Organic solvents used in the invention are selected from the group consisting of C6 to C14 aromatic hydrocarbons, C1 to C5 aliphatic hydrocarbons, C1 to C5 alcohols, C2 to C7 ethers, C1 to C7 acids, halogenated hydrocarbons, C1 to C5 organic acids, and mixtures thereof. Preferably, the organic solvent is selected from a group consisting of C6 to C10 substituted aromatic hydrocarbons, C1 to C5 aliphatic hydrocarbons, halogenated hydrocarbons, cyclic ethers, ketones, esters, nitriles, C4 to C6 straight, branched, or cyclic hydrocarbons, dioxanes, DMF, DMSO, and mixtures thereof.

Halogenated hydrocarbons useful in the present invention are preferably selected from a group consisting of cyclic or acyclic, saturated or unsaturated, aliphatic, or aromatic hydrocarbons. Examples of halogenated hydrocarbons include, but are not limited to, halogenated alkanes such as chloromethane, dichloromethane, chloroethane, dichlorotrifluoroethane, difluoroethane, hexachloroethane, pentafluoroethane, halogenated alkenes, such as tetrachloroethene, dichloroethene, trichloroethene, vinyl chloride, chloro-1,3-butadiene, chlorotrifluoroethylene, or halogenated benzenes such as benzotrichloride, benzyl chloride, bromobenzene, chlorobenzene, chlorotoluene, dichlorobenzene, fluorobenzene, or trichlorobenzene. The preferred halogen is chlorine. The preferred halogenated hydrocarbons are aromatic hydrocarbons or C1 to C4 alkanes. The more preferred halogenated hydrocarbons are chlorobenzene, p-dichlorobenzene, dichloromethane, or o-chlorotoluene. The most preferred halogenated hydrocarbon is dichloromethane and dichloroethane.

Aromatic hydrocarbons useful in the present application are C5 to C14 aromatic hydrocarbons. The preferred aromatic hydrocarbons are toluene and xylene.

Aliphatic cyclic hydrocarbons useful in the present invention are preferably selected from C3 to C8 aliphatic cyclic hydrocarbons, and, more preferably, the aliphatic cyclic hydrocarbons used are cyclohexane and cyclopentane. Ethers useful in the present invention are preferably selected from acyclic and cyclic ethers. Acyclic ethers include alkyl ethers, arylalkyl ethers. Wherein the substituents are those described above for the alkyl and arylalkyl groups. For example acyclic ethers may include diethyl ether, dipropyl ether, isopropyl ether, methyl-tertbutylether, methyl propyl ether, dibutyl ether, ethylene glycol dimethyl ether, dimethoxyethane, bis-methoxymethyl ether, and the like. Cyclic ethers include dioxane, tetrahydrofuran, tetrahydropyran, propyleneoxide, phenyloxirane(styrene oxide), cis-2-butene-oxide(2,3-dimethyloxirane),3-chlorotetrahydrofuran, 2,6-dimethyl-1,4-dioxane, and the like. The preferred ethers are diethyl ether, methyl tertiary butyl ether, and THF.

Inorganic non-aqueous solvents useful in the present invention are preferably selected from a group consisting of liquid ammonia, liquid sulfur dioxide, sulfuryl chloride, and sulfuryl chloride fluoride, phosphoryl chloride, dinitrogen tetroxide, antimony trichloride, bromine pentafluoride, hydrogen fluoride, pure sulfuric acid, and other inorganic acids.

Phase Transfer Catalysts useful in the present invention are preferably are selected from a group consisting of ammonium salts, such as tricaprylylmethylammonium chloride (ALIQUAT® 336), tetra-n-butylammonium bromide (“TBAB”), benzyltriethylammonium chloride (“TEBA”), cetyltrimethylammonium bromide, cetylpyridinium bromide, N-benzylquininium chloride, tetra-n-butylammonium chloride, tetra-n-butylammonium hydroxide, tetra-n-butylammonium iodide, tetra-ethylammonium chloride, benzyltributylammonium bromide, benzyltriethylammonium bromide, hexadecyltriethylammonium chloride, tetramethylammonium chloride, hexadecyltrimethyl ammonium chloride, and octyltrimethylammonium chloride. A preferred phase transfer catalyst is ALIQUAT® 336, TBAB, TEBA, and mixtures thereof; the most preferred being ALIQUAT® 336.

As used herein, the term “Bases” refers to organic and inorganic bases. Inorganic bases useful in the present invention are preferably selected from a group consisting of alkali metal carbonates, alkali metal bicarbonates, and alkali metal hydroxides. Alkali metal carbonates are preferably selected from a group consisting of potassium bi/carbonate, sodium bi/carbonate, cesium carbonate, and hydroxides, such as sodium hydroxide and cesium hydroxide. The preferred inorganic base is sodium carbonate.

Organic bases useful in the present invention are preferably selected from a group consisting of mono-, di-, and tri-(C₁ to C₄ alkyl) amines, such as N,N-dimethylaniline, and N,N-diisopropyl ethyl amine. The preferred organic base is a Tertiary amine.

Certain embodiments of the present application can be depicted in scheme 1 provided below:

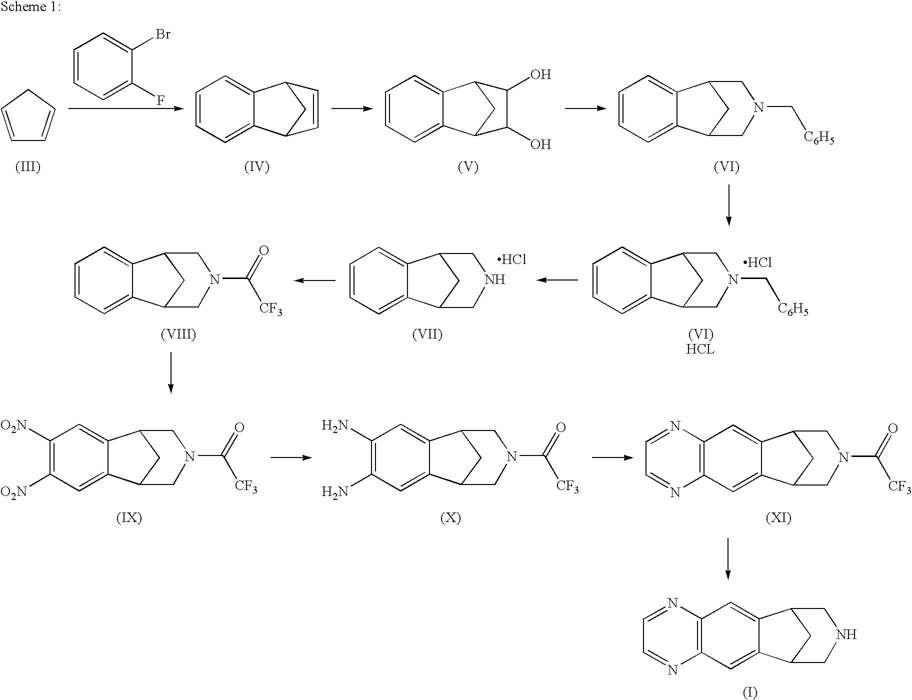

The present invention provides a process for the preparation of Varenicline, the compound of formula (I), as illustrated in Scheme 1, comprising the following steps:

a) In an inert atmosphere, adding a di-halo substituted benzene and a haloalkane in the presence of a solvent to cyclopentadiene (III), over a time period of about 2 to 4 hours, at a temperature of about 50° to 70° C. to obtain the compound of formula (IV), 1,4-Dihydro-1,4-methano-naphthalene in a Grignard reaction;

b) Adding a catalyst in presence of a solvent to compound (IV) of step (a), and subsequently treating compound (IV) with an oxidizing agent over a time period of 2 to about 8 hours, at a temperature of 65° to about 70° C., providing the compound of formula (V), 1,2,3,4-Tetrahydro-1,4-methano-naphthalene-2,3-diol;

c) Adding a suitable oxidizing agent and phase transfer catalyst and a protecting agent, along with a suitable reducing agent to the compound (V) of step (b), providing the compound of formula (VI), 10-Benzyl-10-aza-tricyclo[6.3.1.0^{2,7}]dodeca-2(7),3,5-triene, followed by addition of HCl to obtain the compound 10-Benzyl-10-aza-tricyclo[6.3.1.0^{2,7}]dodeca-2(7),3,5-triene HCl, and, subsequently, debenzylating to provide the compound of formula (VII), 10-Aza-tricyclo[6.3.1.0^{2,7}]dodeca-2(7)3,5-triene hydrochloride;

d) Adding a solvent along with a fluorinating agent, preferably trifluoroacetic anhydride, to the compound of formula (VII) of step (c), to provide the compound of formula (VIII), 1-(10-Aza-tricyclo[6.3.10^{2,7}]dodeca-2 (7),3,5-triene-10-yl)-2,2,2-trifluoro-ethanone;

e) Adding a nitrating source in the presence of a suitable solvent and a Lewis acid to the compound of formula (VIII) of step (d) to obtain the compound (IX), 1-(4,5-Dinitro-10-Aza-tricyclo[6.3.1.0^{2,7}]dodeca-2(7),3,5-triene-10-yl)-2,2,2-trifluoro-ethanone, which is subsequently reduced by hydrogenation to obtain the compound of formula (X), 1-(4,5-Diamino-10-aza-tricyclo[6.3.1.0^{2,7}]dodeca-2(7),3,5-triene-10-yl)-2,2,2-trifluoro-ethanone;

f) Cyclising the compound of formula (X) of step (e) using a 40 percent aqueous glyoxal solution, to obtain the compound of formula (XI), 1-(5,8,14-Triazatetracyclo[10.3.1.0^{2,11}.0^{4,9}]hexadeca-2(11),3,5,9-pentaene)-2,2,2-trifluoro-ethanone, which is deprotected to form the compound of formula (I), Varenicline base that could be further converted to Varenicline L-tartrate.

The present invention provides a process for the preparation of compound IV, 1,4-Dihydro-1,4-methano-naphthalene, according to step (a):

a) In an inert atmosphere, adding a di-halo substituted benzene and a haloalkane in a presence of a solvent over a time period of 2 to about 4 hours, at a temperature range of 50° to about 70° C. to the compound cyclopentadiene (III), to provide the compound of formula (IV).

The di-halo substituted benzene used in step (a) is preferably 1-bromo-2-fluorobenzene. The haloalkane is used in the process of the invention for the initiation of the Grignard reaction in the preparation of the compound of formula (IV). 1,2-dibromoethane is used as a haloalkane. The reaction mass is quenched with chilled water, controlling the exothermicity of the reaction.

The solvent used in step (a) is preferably an ether. More preferably, the ether is cyclic, such as dioxane and tetrahydrofuran, or straight chain, such as diethyl ether and methyl tertiary butyl ether. Most preferably, the ether is tetrahydrofuran.

The present invention also provides a process for the preparation of compound of formula (V), 1,2,3,4-tetrahydro-1,4-methano-naphthalene-2,3-diol, according to step (b):

b) Adding a catalyst in presence of a solvent to compound (IV) of step (a), and subsequently treating compound (IV) with an oxidizing agent over a time period of 2 to about 8 hours, at a temperature of 650 to about 70° C., to provide the compound of formula (V).

The catalyst used in step (b) is preferably selected from a group consisting of heterocyclic amine oxides and morpholine derivatives. More preferably, the catalyst is N-methyl morpholine N-oxide.

The solvents used in step (b) are preferably selected from a group consisting of acetone, n-butanol, toluene, and water.

The oxidizing agents are preferably selected from the group consisting of hypochlorite and other hypohalite compounds, iodine and other halogens, chlorite, chlorate, perchlorate, and other analogous halogen compounds, permanganate salts, ammonium cerium (iv) nitrate and related Cerium (IV) compounds, hexavalent chromium compounds, such as chromic and dichromic acids and chromium trioxide, pyridinium chlorochromate (PCC), and chromate/dichromate compounds, peroxide compounds, Tollen's Reagent, sulfoxides, persulfuric acid, ozone, osmium tetroxide (OsO₄), nitric acid, and nitrous oxide (N₂O). Preferably, the oxidizing agent is osmium tetroxide.

Preferably the oxidizing agent is used at the reflux temperature of the relevant solvent.

Preferably the osmium tetroxide used in step (b) is recycled.

The reaction in step (b) requires about 1 to about 4 hours, and, preferably, about 2 to about 3 hours.

The present invention also provides a process for the preparation of compound VI, 10-Benzyl-10-aza-tricyclo[6.3.1.0^{2,7}]dodeca-2(7),3,5-triene according to step (c):

(c) Adding a suitable oxidizing agent and phase transfer catalyst and a protecting agent, along with a suitable reducing agent to the compound (V) of step (b) to obtain the compound of formula (VI).

The diol compound of formula (V) used in step (c) is combined with biphasic solvents, such as methylene dichloride and water, at a temperate of about −5° C. to about 10° C. The oxidizing agent selected from the group described above and a phase transfer catalyst are added, and the temperature is maintained at about −5° to about 10° C. Preferably, the oxidizing agent used in the process is sodium periodate. Preferably, the phase transfer catalyst used in the process is Benzyl triethyl ammonium chloride.

The N-protecting agents used in the process are selected from the group consisting of Carbobenzyloxy (Cbz) group, tert-Butyloxycarbonyl (BOC) group, 9-fluorenylmethyloxycarbonyl (FMOC) group Benzyl (Bn) group, and p-methoxyphenyl (PMP) group. Preferably, the protecting agent used contains a benzyl group. More preferably, the protecting agent used is benzyl amine. The reducing agent is preferably selected from a group consisting of ferrous ion, Lithium aluminum hydride (LiAlH₄), nascent hydrogen, sodium amalgam, sodium borohydride (NaBH₄), stannous ion, sulfite compounds, hydrazine, zinc-mercury amalgam (Zn(Hg), diisobutylaluminum hydride (DIBAH), Lindlar catalyst, and oxalic acid (C₂H₂O₄). More preferably, the reducing agent is sodium borohydride in the presence of acetic acid.

The present invention describes a process for the preparation of compound (X) 1-(4,5-Diamino-10-aza-tricyclo[6.3.1.0^{2,7}]dodeca-2(7),3,5-triene-10-yl)-2,2,2-trifluoro-ethanone according to step (e):

(e) Compound (IX), 4,5-Dinitro-10-Aza-tricyclo-[6.3.10^{2,7}]dodeca-2 (7),3,5-triene-10-yl)-2,2,2-trifluoro-ethanone is reduced by hydrogenation to obtain the compound of formula (X).

The obtained Compound (X) is further cyclised according to the following step (f):

(f) Cyclising the compound of formula (X) of step (e) using 40 percent aqueous Glyoxal solution to obtain the compound of formula (XI) which is deprotected to form the desired compound of formula (I), Varenicline base that can be further converted to Varenicline L-Tartrate.

The present invention also provides an in-situ process for the preparation of the compound of formula (IX) from the compound of formula (V). The in-situ process is a one-pot reaction that provides the compound of formula (IX) in a significantly shorter reaction time than is possible with prior art processes. In addition, the use of the in-situ process does not require the isolation of intermediates, reducing the cost of reagents. Therefore, altogether the use of the in-situ process enables preparation of the compound of formula (IX) in a more cost effective way.

The in-situ process of the present invention is described according to scheme 2 depicted below:

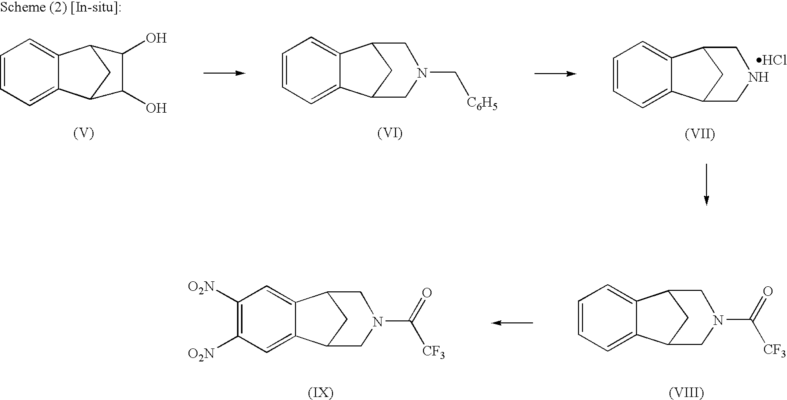

The present invention also provides an in-situ process for the preparation of the compound of formula (IX), 1-(4,5-Dinitro-10-Aza-tricyclo[6.3.1.0^{2,7}]dodeca-2(7),3,5-triene-10-yl-2,2,2-trifluoro ethanone from the compound of formula (V), 1,2,3,4-tetrahydro-1,4-methano-napthalene-2,3-diol, comprising the following steps:

a) Adding a phase transfer catalyst, a suitable oxidizing agent, a protecting agent, a suitable base, and a reducing agent to the compound of formula (V) and a biphasic solvent, providing the compound of formula (VI), followed by the preparation of the haloacid salt of compound of formula (VI) HCl,

b) Without isolating the compound of formula (VI) HCl of step (a), adding, in the same pot as step (a), a suitable solvent and a debenzylating agent under pressure to obtain the compound of formula (VII), followed by the in-situ formation of the haloacid salt of the compound of formula (VII) in the presence of a suitable solvent and a combination of the same with a suitable halo-acid to obtain the in-situ formation of haloacid salt of the compound of formula (VII) HCl salt,

c) Without isolating the compound of formula (VII) HCl, adding a suitable solvent and base in the presence of a reactant to the same pot as that of step (b), providing a compound of formula (VIII), which is further treated for workup,

d) Without isolating the compound of formula (VIII), adding solvent to the same pot as that of step c), and treating the organic layer with a nitrating source to provide the desired compound of formula (IX).

In one embodiment, the present invention also provides a process for preparing a purified compound of formula (VI), 10-Benzyl-10-aza-tricyclo[6.3.1.0^{2,7}]dodeca-2(7), 3,5-triene, comprising combining or contacting the compound of formula (VI) with water and an alkali metal dihydrogen phosphate. The alkali metal dihydrogen phosphate used in the process described above is selected from the group consisting of potassium dihydrogen phosphate and sodium dihydrogen phosphate. Most preferably, potassium dihydrogen phosphate is used in the process.

Preferably, about 5 percent to about 25 percent of an aqueous solution of potassium dihydrogen phosphate is used, more preferably, about 8 percent to about 15 percent is used, and, most preferably, 10 percent of an aqueous solution of potassium dihydrogen phosphate is used in the process. Preferably the compound of formula (VI) is washed with the aqueous solution of alkali metal dihydrogen phosphate.

The water used in the above described process is preferably demineralized water (DM water).

The obtained purified compound of formula VI is preferably further recovered by concentration using reduced pressure, preferably at a temperature of about 35° C. to about 40° C.

The compound of formula (VI) obtained according to the process described above has a purity of about 95 percent to about 97 percent by area HPLC, and, more preferably it is about 96.4 percent.

In another embodiment, the present invention also provides a process for preparing a purified compound of formula (XI), 1-(5,8,14-Triazatetracyclo[10.3.1.0^{2,11}.0^{4,9}]hexadeca-2(11),3,5,9-pentaene)-2,2,2-trifluoro-ethanone, comprising combining the compound of formula (XI) and an acid. Preferably, the compound of formula (XI) is washed with the acid or maintained in solution or suspension with the acid.

The acid used in the process describe above may be a mineral acid or an organic acid. The mineral acid is preferably selected from the group consisting of hydrochloric acid, hydrobromic acid, hydroiodic acid, sulfuric acid, and mixtures thereof. Preferably, the mineral acid used is hydrochloric acid or sulfuric acid, and, more preferably, is hydrochloric acid. The organic acid that may be used in the process described above is selected from the group consisting of lactic acid, acetic acid, formic acid, citric acid, oxalic acid, and mixtures thereof. Preferably, the organic acid used is acetic acid or formic acid. Most preferably, the acid used in the above described process is hydrochloric acid.

The acid is preferably used at a concentration of about 0.5N to about 2N, more preferably, at about 0.5N to about 1N, and, even more preferably, at about 1.5N. Preferably, the acid is added at a temperature of about 35 to about 45° C. Preferably, following the addition of acid, the reaction mixture described above has a pH of about 0.5 to about 1.

Optionally, the acid is added in stages until obtaining the desired pH.

Preferably, water is used in the process described above, and, more preferably, the water is DM water.

Optionally, aqueous sodium chloride solution is added to the organic layer to remove traces of water, and to obtain the purified compound of formula (XI).

The obtained purified compound of formula XI is preferably further recovered by concentration and using reduced pressure and filtration.

Preferably concentration is performed at a temperature of about 35° C. to about 40° C.

Optionally, n-heptane is added following the concentration step.

The compound of formula (XI) obtained according to the process described above is obtained with a purity of about 98 percent to about 100 percent by area HPLC, more preferably it is about 98.5 percent to about 99.5 percent, and, most preferably, about 99 percent.

In yet another embodiment, the present invention provides a process for preparing Varenicline comprising:

a) Adding a di-halo substituted benzene in the presence of a solvent and a haloalkane to cyclopentadiene to obtain a compound of formula (IV), 1,4-Dihydro-1,4-methano-naphthalene in a Grignard reaction;

b) Treating the compound of formula (IV) obtained in step (a) with a catalyst in the presence of a solvent and, subsequently, an oxidizing agent to obtain a compound of formula (V), 1,2,3,4-tetrahydro-1,4-methano-naphthalene-2,3 diol;

c) Adding an oxidizing agent, a phase transfer catalyst, a protecting agent, and a reducing agent to the compound of formula (V) obtained in step (b) to obtain a compound of formula (VI), 10-Benzyl-10-aza-tricyclo[6.3.1.0^{2,7}]dodeca-2(7),3,5-triene, and, optionally, purifying the compound of formula VI in a process step, comprising contacting the compound of formula VI with water and an alkali metal dihydrogen phosphate;

d) Adding HCl to the compound of formula VI obtained in step (c) to obtain a compound 10-Benzyl-10-aza-tricyclo[6.3.1.0^{2,7}]dodeca-2(7), 3,5-triene HCl, and, subsequently, debenzylating the 10-Benzyl-10-aza-tricyclo[6.3.1.0^{2,7}]dodeca-2(7),3,5-triene HCl to obtain a compound of formula (VII), 10-Aza-tricyclo[6.3.1.0^{2,7}]dodeca-2(7)3,5-triene hydrochloride;

e) Adding a solvent and a fluorinating agent to the compound of formula (VII) obtained in step (d) to obtain a compound of formula (VIII), 1-(10-Aza-tricyclo[6.3.1.02]dodeca-2 (7), 3,5-triene 10-yl)-2,2,2-trifluoro-ethanone;

f) Adding a nitrating source in the presence of a solvent and a Lewis acid to the compound of formula (VIII) obtained in step (e), to obtain a compound of formula (IX), 1-(4,5-Dinitro-10-Aza-tricyclo[6.3.1.0^{2,7}]dodeca-2(7), 3,5-triene-10-yl)-2,2,2-trifluoroethanone, and, subsequently, reducing the compound of formula (IX) by hydrogenation to obtain a compound of formula (X), 1-(4,5-Diamino-10-aza-tricyclo[6.3.1.0^{2,7}]dodeca-2(7),3,5-triene-10-yl)-2,2,2-trifluoro-ethanone.

g) Cyclising the compound of formula (X) obtained in step (f) with a 40 percent aqueous glyoxal solution to obtain a compound of formula (XI), 1-(5,8,14-Triazatetracyclo[10.3.1.0^{2,11}.0^{4,9}]hexadeca-2(11),3,5,9-pentaene)-2,2,2-trifluoro-ethanone; and purifying the compound of formula XI in a process step, comprising combining or contacting the compound of formula XI with an acid; and

h) Deprotecting the compound of formula XI obtained in step (g) to obtain Varenicline.

In one embodiment, the present invention further provides a process for preparing Varenicline L-tartrate comprising, obtaining Varenicline base according to the process described above, and converting the obtained Varenicline base to Varenicline L-tartrate.

Conversion of Varenicline base to Varenicline L-Tartrate may be obtained according to methods known in the art, such as the one described in U.S. Pat. No. 6,890,927, incorporated herein by reference, wherein L-tartaric acid in methanol is combined with Varenicline base in methanol.

Having thus described the invention with reference to particular preferred embodiments and illustrative examples, those in the art may appreciate modifications to the invention as described and illustrated that do not depart from the spirit and scope of the invention as disclosed in the specification. The Examples are set forth to aid in understanding the invention, but are not intended to, and should not be construed to limit the scope of the invention in any way.

### EXAMPLES

HPLC: HPLC methodology for analyzing the compounds of formula IV, formula V, formula VI, formula VII, and formula IX.
Buffer: Prepared 0.02M Potassium dihydrogen orthophosphate in water, where the pH was adjusted to 3.5 with an H₃PO₄ solution.

### Mobile Phase:

### Eluent A: Buffer:Methanol (50:50)

### Eluent B: Eluent A:Acetonitrile (35:65)

### Diluent: Water:Acetonitrile (50:50)

### Chromatographic Conditions

| | |
|---|---|
| Column: | Ascentis Express C18 150 × 4.6 mm, |
| | 2.7μ P/N 53829-U or equivalent. |
| Flow | 0.4 ml/min |
| Injection Volume | 5 μl |
| Detector | 215 nm |
| Column temperature | 30° C. |
| Autosampler | 10° C. |
| temperature | |
| Run time | 40 minutes |

HPLC methodology for analyzing the compound of formula VII

### Buffer

Prepared 0.02M Potassium dihydrogen orthophosphate in water, where the pH was adjusted to 3.5 with an H₃PO₄ solution.

### Mobile Phase:

### Eluent A: Buffer:Methanol (70:30)

### Eluent B: Eluent A:Acetonitrile (35:65)

### Diluent: Eluent-A

Chromatofraphic conditions

| | |
|---|---|
| Column: | Ascentis Express C18 150 × 4.6 mm, |
| | 2.7μ P/N 53829-U or equivalent. |
| Flow | 0.4 ml/min |
| Injection Volume | 5 μl |
| Detector | 215 nm |
| Column temperature | 30° C. |
| Autosampler | 10° C. |
| temperature | |
| Run time | 40 minutes |

HPLC methodology for analyzing the compound of formula X

Buffer: Prepared 0.02M Ammonium dihydrogen orthophosphate in water, where the pH was adjusted to 6.0 with an ammonia solution.

### Mobile Phase:

### Eluent A: Buffer

### Eluent B: Methanol

### Diluent: Acetonitrile

### Chromatographic Conditions

| | |
|---|---|
| Column: | Inertsil ODS 3V 150 × 4.6 mm, |
| | 5μ P/N 5020-01801 or equivalent. |
| Flow | 1.0 ml/min |
| Injection Volume | 5 μl |
| Detector | 210 nm |
| Autosampler | 10° C. |
| temperature | |
| Run time | 40 minutes |

HPLC methodology for analyzing the compound of formula XI.

Buffer: Prepared 0.02M Ammonium dihydrogen orthophosphate in water, where the pH was adjusted to 6.0 with an ammonia solution.

### Mobile Phase:

### Eluent A: Buffer

### Eluent B: Acetonitrile

### Diluent: Acetonitrile

### Chromatographic Conditions

| | |
|---|---|
| Column: | Inertsil ODS 3V 150 × 4.6 mm, |
| | 5μ P/N 5020-01801 or equivalent. |
| Flow | 1.0 ml/min |
| Injection Volume | 5 μl |
| Detector | 210 nm |
| Column temperature | 30° C. |
| Autosampler | 10° C. |
| temperature | |
| Detector run time | 30 minutes |
| Equilibration time | 5 minutes |

HPLC methodology for analyzing Varenicline base (formula I)—

### Mobile Phase:

Eluent A: 80%-0.02M Ammonium acetate adjusted to pH=8.5 with diluted NH₄OH
10%—MeOH
10%—ACN
   Eluent B: 20%-0.02M Ammonium acetate adjusted to pH=8.5 with diluted NH₄OH
50%—MeOH
30%—ACN

### Diluent: Eluent A

### Chromatographic Conditions

| | |
|---|---|
| Column: | Purospher STAR RP-18e 75, 4 mm, 3um. |
| | CN 1.51460.0001 or equivalent. |
| Flow | 1.0 ml/min |
| Injection Volume | 10 μl |
| Detector | 235 nm |
| Column temperature | 25° C. |
| Run time | 13 minutes |
| Equilibration time | 5 min |

### Example 1

### Preparation of 1,4-Dihydro-1,4-methano-napthalene (IV)

Magnesium turnings (85.1 g) and few iodine crystals were stirred under a nitrogen atmosphere in anhydrous THF (482.5 ml) in a clean, dry round bottomed flask with an addition funnel. The reaction mass was stirred and warmed to reflux (i.e., 60° to 65° C.) by a removable heating mantle. To this, 1-Bromo-2-fluoro benzene (21.2 gm) was added, followed by 1,2-dibromoethane (3.86 ml) to initiate the Grignard reaction. The heating source was removed, and reflux was maintained by the addition of mixture of 1-Bromo-2-fluoro benzene (500 g) and cyclopentadiene (193 g) (Mixture temperature 0° to 5° C.) within 2.5 to 3.5 hours. Reflux temperature was maintained for 2.5 hours. Progress of reaction was monitored by HPLC. The reaction mass was quenched in ice chilled water, and charged with concentrated HCl to obtain a clear solution. The product was extracted with hexane (500 ml×1, 482.5 ml×4). The combined organic layer was washed with 8 percent aqueous sodium bicarbonate solution (482.5 ml). The organic layer organic layer was concentrated to obtain an oil product, which was further purified by high vacuum distillation (2-10 mmHg) at vapour temperature of 500 to 55° C. to obtain the compound of formula IV (1,4-Dihydro-1,4-methano-napthalene) (262.3 gm, HPLC purity 90.20%).

### Example 2

### Procedure for Preparation of 1,2,3,4-Tetrahydro-1,4-methano-napthalene-2,3-diol (V)

In a 1 l 4-neck round bottom flask equipped with mechanical stirrer was placed N-methyl morpholine N-oxide (401.9 g) and 1,4-dihydro-1,4-Methanonaphtalen-9-ylidene (414.0 g) in acetone (828 ml) and water (105 ml) at 25° to 30° C. To this mixture was added 15 mole percent solution of osmium tetroxide (1.0 g) in n-butanol (26 ml) at 25° to 30° C. The reaction mixture was stirred at reflux temperature, i.e., 65° to 70° C. for 2 hours. Progress of reaction monitored by HPLC. Distilled out reaction mixture under vacuum below 80° C., charged acetone (1035 ml) into the crude product, and stirred for 1.0 hour at 25° to 30° C. The reaction mass was filtered and washed with acetone (620 ml). The product was air dried to obtain a first crop of 1,2,3,4-Tetrahydro-1,4-methano-napthalene-2,3-diol (412.38 g, HPLC purity 99.85 percent.).

### Example 3

### Procedure for Preparation of 1,2,3,4-Tetrahydro-1,4-methano-napthalene-2,3-diol (V)

In a 1 l 4-neck round bottom flask equipped with mechanical stirrer was placed N-methyl morpholine N-oxide (521.35 g) and 1,4-dihydro-1,4-methanonaphtalen-9-ylidene (537.0 g) in the mother liquor of the previous reaction (2000 ml) water (70 ml) at 250 to 30° C. Approximately 1000 ml of acetone was distilled out at 65° to 70° C. The reaction mixture was stirred at reflux temperature, i.e., 65° to 70° C. for 3 hours. Progress of reaction monitored by HPLC. The reaction mixture was distilled out under vacuum below 80° C. The crude product was charged with acetone (1074 ml), and stirred for 1.0 hour at 25° to 30° C. The reaction mass was filtered and washed with acetone (805 ml), and the product was air dried to obtain a first crop of 1,2,3,4-Tetrahydro-1,4-methano-napthalene-2,3-diol (552.0 g, HPLC purity 99.94 percent). The mother liquor was concentrated to provide a semi-solid that was then triturated with acetone (1074 ml), and stirred for 1 hour. The precipitated solid was filtered and washed with acetone (805 ml). The product was air dried to provide a second crop of 1,2,3,4-Tetrahydro-1,4-methano-napthalene-2,3-diol (30.0 g HPLC purity 99.22 percent).

### Example 4

### Comparative Example

### Procedure for Preparation of 10-Benzyl-10-aza-tricyclo[6.3.1.0^{2,7}]dodeca-2(7),3,5-triene (VI)

1,2,3,4-Tetrahydro-1,4-methano-napthalene-2,3-diol (100 g) was combined with methylene dichloride (MDC) (1000 ml) and DM water (2.6 l) in a suitable round bottom flask, having cool water bath (approximately 10° C.). To this, sodium periodate (127.6 g) and Benzyl Triethyl ammonium chloride (1.3 g) were added at 10° to 15° C. The resulting mixture was stirred for 1 hour at 20° to 25° C. Progress of reaction was monitored by TLC (ethyl acetate:hexane (5:5)). The layers were separated, and the aqueous layer was extracted with MDC (500 ml). The combined organic layer was washed with DM water (2×500 ml), dried over sodium sulfate (10 g), and collected in a round bottom flask (mixture A). Sodium triacetoxy borohydride (301 g) in MDC (1540 ml) was charged in a separate dry round bottom flask, and cooled to 0° to 5° C. (mixture B). After mixture B was prepared, benzyl amine (63.9 g) was added to mixture A, and the resulting mixture A was stirred for 2 minutes. The benzyl amine should only be charged in the MDC layer of mixture A after the sodium triacetoxy borohydride suspension of mixture B is prepared. Mixture A was then added to mixture B.

Without delay, the resulting mixture was allowed to warm to room temperature, and stirred for 1.5 hours at 20° to 25° C. Progress of the reaction was monitored by TLC (ethyl acetate:hexane (5:5)). The reaction was quenched by adding 10 percent aqueous sodium carbonate solution to adjust the pH to 8 to 9, and the mixture was stirred for 1 hour at 20° to 25° C. The layers were separated, and the aqueous layer was extracted with MDC (500 ml). The combined organic layer was washed with 10 percent aqueous potassium dihydrogen phosphate (2×1 l) at 20° to 30° C., followed by a wash with DM water (1×0.5 l), the layers were separated, and the organic layer was concentrated. Following the addition of DM water (1×0.5 l), concentration at reduced pressure at about 35° to 40° C. provided an oil product, i.e., 10-Benzyl-10-aza-tricyclo[6.3.1.02,7]dodeca-2(7),3,5-triene (137 g, HPLC purity: 96.4%)

### Example 5

### Procedure for Preparation of 10-Benzyl-10-aza-tricyclo[6.3.1.0^{2,7}]dodeca-2(7),3,5-triene (VI)

1,2,3,4-Tetrahydro-1,4-methano-napthalene-2,3-diol (5 g) was combined with MDC (50 ml) and DM water (130 ml) with in a suitable round bottom flask, having a cool water bath (approximately 10° C.). To this, sodium periodate (6.3 g) and benzyl triethyl ammonium chloride (0.6 g) were added at 10° to 15° C. The resulting mixture was stirred for 1 hour at 20° to 25° C. Progress of reaction was monitored by TLC (ethyl acetate:hexane (5:5)). The layers were separated, and the aqueous layer was extracted with MDC (2×50 ml). The combined organic layer was washed with DM water (4×50 ml), dried over sodium sulfate (5 g), and collected in a round bottom flask (mixture A). In a separate dry round bottom flask, sodium borohydride (1.06 g) and acetic acid (5.1 g) were charged in MDC (50 ml), and cooled to 0° to 5° C. (mixture B). After mixture B was prepared, benzyl amine (3.2 g) was added to mixture A, and the resulting mixture A was stirred for 2 minutes. The benzyl amine should only be charged in the MDC Layer of mixture A after the sodium triacetoxy borohydride suspension of mixture B is prepared. Mixture A was then added to mixture B.

Without delay, the resulting mixture was allowed to warm to room temperature, and stirred for 1.0 hour at 200 to 25° C. Progress of reaction was monitored by TLC (ethyl acetate:hexane (5:5)). The reaction was quenched by adding 30 percent aqueous sodium carbonate solution (30 ml) to adjust the pH to 8 to 9, and the mixture was stirred for 1 hour at 20° to 25° C. The layers were separated, and the aqueous layer was extracted with MDC (2×500 ml). The combined organic layer was washed with DM water (1×50 ml). Concentration provided an oil product, i.e., 10-Benzyl-10-aza-tricyclo[6.3.1.02,7]dodeca-2(7),3,5-triene (8.7 g).

### Example 6

### Procedure for Preparation of 10-Aza-tricyclo[6.3.1.0^{2,7}]dodeca-2(7)3,5-triene hydrochloride(VII)

Methanolic hydrochloride (116.6 g, 13.8%) was added at 0° to 5° C. to a suspension of 10-Benzyl-10-aza-tricyclo[6.3.1.0 2,7]dodeca-2(7),3,5-triene (100 g) in methanol (600 ml). The resulting solution was hydrogenated under a hydrogen pressure of 5 to 6 kg/cm² over 20 percent palladium hydroxide (26 g) at 20° to 40° C. for 3 to 6 hours. The reaction was monitored by HPLC, then filtered trough a celite pad, and the solvent was distilled out under vacuum. To this residue, MDC (500 ml) was added, and the solvent was completely distilled out under vacuum to provide the crude product, which was further purified using acetone (450 ml) to obtain the pure product, i.e., 10-Aza-tricyclo[6.3.1.0^{2,7}]dodeca-2(7)3,5-triene hydrochloride (HPLC Purity 98.23 percent, yield 89.24 percent)

### Example 7

### Comparative Example

### Procedure for Preparation of 1-(10-Aza-tricyclo[6.3.1.0^{2,7}]dodeca-2(7),3,5-triene-10-yl)-2,2,2-trifluoro ethanone (Compound VIII)

A suspension of 10-Aza-tricyclo[6.3.1.02,7]dodeca-2(7),3,5-triene hydrochloride (127 g) and MDC (2032 ml) was stirred at 0° to 5° C., and treated with triethylamine (157.8 g). To this solution, tri-fluoro acetic anhydride (163.7 g) was added at 0° to 5° C. over a period of 30 to 40 minutes. The resulting solution was allowed to warm to room temperature, and stirred for 2 hours. The progress of the reaction was checked by TLC (ethyl acetate:hexane:ammonia (7:3:1 drop)). The reaction was quenched by the addition of DM water (952.5 ml) at 5° to 10° C., and stirred for a half hour. The layers were separated, and the aqueous layer was extracted with MDC (508 ml). The organic layer was washed with 1N HCl (3×762 ml) and 10 percent aqueous sodium chloride solution (508 ml). Concentration provided a 1-(10-Aza-tricyclo[6.3.1.02,7]dodeca-2(7),3,5-triene-10-yl)-2,2,2-trifluoro ethenone product (158.75 g, HPLC purity 96.76 percent).

### Example 8

### Procedure for preparation of 10-Aza-tricyclo[6.3.1.0^{2,7}]dodeca-2(7),3,5-triene hydrochloride (VII) Process

A suspension of 10-Aza-tricyclo[6.3.1.0^{2,7}]dodeca-2(7),3,5-triene hydrochloride (500 g) and MDC (8 l) was stirred at 0° to 5° C., and treated with triethylamine (621.4 g). To this solution, tri-fluoro acetic anhydride (645 g) was added at 0° to 5° C. over 30 to 40 minutes. The resulting solution was allowed to warm to room temperature, and stirred for 2 hours. The progress of the reaction was checked by HPLC. The reaction was quenched by addition of DM water (3.75 l) at 5° to 10° C., and stirred for 1.0 hour. The layers were separated, and the aqueous layer was extracted with MDC (1 l). The organic layer was washed with 1N HCl (3×3 l) and 10 percent aqueous sodium chloride solution (2 l). Concentration of the organic layer to 2 l provided a 1-(10-Aza-tricyclo[6.3.1.0^{2,7}]dodeca-2(7),3,5-triene-10-yl)-2,2,2-trifluoro ethenone product, which was further processed as described below stage (HPLC purity 98.12 percent).

### Example 9

### Comparative Example

### Procedure for Preparation of 1-(4,5-Dinitro-10-aza-tricyclo[6.3.1.0^{2,7}]dodeca-2(7),3,5-triene-10-yl)-2,2,2-trifluoro-ethanone

Fuming nitric acid (311.33 g) was added over 25 to 35 minutes at 0° to 5° C. to a solution of Tri-fluoro methane sulfonic acid (1364.49 g) in MDC (2.0 l), and stirred for 10 to 15 minutes. To this resulting mixture 1-(10-Aza-tricyclo[6.3.1.02,7]dodeca-2(7),3,5-triene-10-yl)-2,2,2-trifluoro-ethanone solution [504.0 g in MDC (2.0 l)] was added over 1.0 to 1.5 hours at 0° to 5° C., and the reaction mixture was warmed to room temperature. The reaction mass was stirred at 25° to 30° C. for 2.5 hours. The progress of the reaction was checked by HPLC. The reaction mixture was quenched in DM water (5.0 l) at 0° to 15° C. The layers were separated, and the aqueous layer was extracted with MDC (2×3 l). The combined organic layer was washed with DM water (3×3 l), and then with 8 percent aqueous NaHCO₃ solution (1×2.5 l) and DM water (1×2 l). The organic layer was concentrated to obtain crude 1-(4,5-Dinitro-10-aza-tricyclo[6.3.1.02,7]dodeca-2(7),3,5-triene-10-yl)-2,2,2-trifluoro-ethanone. That product was triturated with ethyl acetate (655 ml) at 55° to 60° C. for 2 hours. The solid was filtered and washed with chilled ethyl acetate (505 ml) to provide pure 1-(4,5-Dinitro-10-aza-tricyclo[6.3.1.02,7]dodeca-2(7),3,5-triene-10-yl)-2,2,2-trifluoro-ethanone with a yield of 430 g and an HPLC purity of 99.69 percent. The solid was dried under vacuum at 450 to 50° C.

### Example 10

### Procedure for Preparation of 1-(4,5-Dinitro-10-aza-tricyclo[6.3.1.0^{2,7}]dodeca-2(7),3,5-triene-10-yl)-2,2,2-trifluoro-ethanone (IX)

Fuming nitric acid (390.2 g) was added over 25 to 35 minutes at 0° to 5° C. to a solution of tri-fluoro methane sulfonic acid (1.7 kg) in MDC (2.52 l). The mixture was stirred for 10-15 minutes. To this resulting organic layer, 1-(10-Aza-tricyclo[6.3.1.0^{2,7}]dodeca-2(7),3,5-triene-10-yl)-2,2,2-trifluoro-ethanone in MDC was added over 1.0 to 1.5 hours at 0° to 5° C. After completion of addition, the temperature was immediately raised to 25° to 30° C. The reaction mass was stirred at 25° to 30° C. for 2.0 hours. The progress of the reaction was checked by HPLC. The reaction mixture was quenched in DM water (6.5 l) at 0° to 5° C. The layers were separated, and the aqueous layer was extracted with MDC (2×1.26 l). The combined organic layer was washed with DM water (3×3.2 l), and then with an 8 percent aqueous NaHCO₃ solution (1×3.2 l) and DM water (1×2.5 l). The organic layer was concentrated to provide crude 1-(4,5-Dinitro-10-aza-tricyclo[6.3.1.0^{2,7}]dodeca-2(7),3,5-triene-10-yl)-2,2,2-trifluoro-ethanone. This was triturated with ethyl acetate (1.30 l) at 55° to 60° C. for 2 hours. The solid was filtered and washed with chilled ethyl acetate (630 ml) to provide pure 1-(4,5-Dinitro-10-aza-tricyclo[6.3.1.0^{2,7}]dodeca-2(7),3,5-triene-10-yl)-2,2,2-trifluoro-ethanone having a yield of 68.9 percent and an HPLC purity of 99.55 percent.

### Example 11

### Comparative Example

### Preparation of 1-(4,5-Diamino-10-aza-tricyclo[6.3.1.0^{2,7}]dodeca-2(7),3,5-triene-10-yl)-2,2,2-trifluoro-ethanone(X)

1-(4,5-Dinitro-10-aza-tricyclo[6.3.1.02,7]dodeca-2(7),3,5-triene-10-yl)-2,2,2-trifluoro-ethanone (100.0 g) was hydrogenated in methanol (1.0 lit) under a hydrogen pressure of 5-6 Kg/cm2 over 20 percent palladium hydroxide (10.0 g) for 4-5 hours. The reaction was monitored by HPLC. The reaction mixture was then filtered through a celite pad, and concentrated to provide 1-(4,5-Diamino-10-aza-tricyclo[6.3.1.02,7]dodeca-2(7),3,5-triene-10-yl)-2,2,2-trifluoro-ethanone (HPLC purity—not less than 98 percent.). The product was dried under vacuum at 400 to 45° C.

### Example 12

### Preparation of 1-(4,5-Diamino-10-aza-tricyclo[6.3.1.0^{2,7}]dodeca-2(7),3,5-trine-10-yl)-2,2,2-trifluoro-ethanone (X)

1-(4,5-Dinitro-10-aza-tricyclo[6.3.1.0^{2,7}]dodeca-2(7),3,5-triene-10-yl)-2,2,2-trifluoro-ethanone (700.0 g) was hydrogenated in methanol (7.0 lit) under a hydrogen pressure of 5-6 Kg/cm² over 20 percent palladium hydroxide (70.0 g) for 4 to 5 hours. The reaction was monitored by HPLC. The reaction mixture was then filtered through a celite pad, and concentrated to provide 1-(4,5-Diamino-10-aza-tricyclo[6.3.1.0^{2,7}]dodeca-2(7),3,5-triene-10-yl)-2,2,2-trifluoro-ethanone (HPLC purity—not less than 98 percent). This was further dissolve in THF (1.4 l), and processed as described below.

### Example 13

### Preparation of 1-(5,8,14-Triazatetracyclo[10.3.1.0^{2,11}.0^{4,9}]hexadeca-2(11),3,5,9-pentaene)-2,2,2-trifluoro-ethanone (XI)

1-(4,5-Diamino-10-aza-tricyclo[6.3.1.02,7]dodeca-2(7),3,5-triene-10-yl)-2,2,2-trifluoro-ethanone was charged in a solution of THF (400 ml) and 40 percent aqueous glyoxal solution (56.0 ml in 280.0 ml DM water) at 25° to 30° C., and then stirred at 55° to 60° C. for 2 hours. The progress of reaction was checked by TLC (MDC:MeOH 9:1). The reaction mass was cooled to 250 to 30° C., and extracted with ethyl acetate (1 l×1, 300 ml×2). The combined organic layer was washed with DM water (3×500 ml), and then with 10 percent aqueous sodium chloride solution (1×500 ml). The organic layer was concentrated to obtain an oil product. i.e., 1-(5,8,14-Triazatetracyclo[10.3.1.02,11.04,9]hexadeca-2(11),3,5,9-pentaene)-2,2,2-trifluoro-ethanone (HPLC purity—not less than 88 percent), which solidified on standing.

### Example 14

### Preparation of 1-(5,8,14-Triazatetracyclo[10.3.1.0^{2,11}.0^{4,9}]hexadeca-2(11),3,5,9-pentaene)-2,2,2-trifluoro-ethanone (XI)

THF (400 ml), 1-(4,5-Diamino-10-aza-tricyclo[6.3.1.02,7]dodeca-2(7),3,5-triene-10-yl)-2,2,2-trifluoro-ethanone (100.0 gm) and DM water (400 ml) at 25° to 30° C. were charged in a clean, dry 3.0 l 4-neck round bottom flask equipped with mechanical stirrer, thermopocket, and addition funnel. Then, a 40 percent aqueous glyoxal solution (56.0 ml in 280.0 ml DM water) at 25° to 30° C. was charged into the resulting solution. The resulting mixture was then stirred at 55° to 60° C. for 2 hours. Progress of the reaction checked by TLC (MDC:MeOH:9:1). The reaction mass was cooled to 25° to 30° C., and extracted successively with ethyl acetate (1 l×1, 300 ml×2). The combined organic layer was washed with 0.5 N HCl (100 ml×2), DM water (3×500 ml), and a 10 percent aqueous sodium chloride solution (1×500 ml). The resultant organic layer was concentrated to provide an oily product. i.e., 1-(5,8,14-Triazatetracyclo[10.3.1.02,11.04,9]hexadeca-2(11),3,5,9-pentaene)-2,2,2-trifluoro-ethanone (yield-83 g, HPLC purity—not less than 98%.), which solidified on standing.

### Example 15

### Preparation of 1-(5,8,14-triazatetracyclo[10.3.1.0^{2,11}.0^{4,9}]hexadeca-2(11),3,5,9-pentaene)-2,2,2-trifluoro-ethanone (XI)

THF (2.3 l, 578 g) was added to a mixture of 1-(4,5-Diamino-10-aza-tricyclo[6.3.1.0^{2,7}]dodeca-2(7),3,5-triene-10-yl)-2,2,2-trifluoro-ethanone solution and DM water (2.3 l) at 25° to 30° C. To the above solution, a 40 percent aqueous glyoxal solution (323.8 ml in 1.7 l DM water) was added at 25° to 30° C. The resultant mixture was then stirred at 55° to 60° C. for 2 hours. Progress of the reaction was checked by HPLC. THF was distilled out completely, and 1.7 l of DM water was charged, and the resulting mixture was extracted with ethyl acetate (5.7 l×1, 1.7 l×2). The combined organic layer was washed with DM water (3×2.8 l). To this organic layer, DM water (2.8 l) was added, and the pH was adjusted to 1.0 to 0.5 with 0.5N HCl at 35° to 45° C. The layer was then stirred for 30 minutes, the layer was separated, and the process was repeated twice. The organic layer was washed with 10 percent aqueous sodium chloride solution (1×2.8 ml) successively. The resultant organic layer was charcolized at reflux temperature, and then further concentrated at reduced pressure at about 35° to 40° C. to obtain a solid product that was filtered in n-Heptane (1.1 l), providing 1-(5,8,14-Triazatetracyclo[10.3.1.0^{2,11}.0^{4,9}]hexadeca-2(11),3,5,9-pentaene)-2,2,2-trifluoro-ethanone (yield-74.48%, HPLC purity—99 percent).

### Example 16

### Comparative Example

### Procedure for Preparation of Varenicline Base (I)

A suspension of 1-(5,8,14-Triazatetracyclo[10.3.1.02,11.04,9]hexadeca-2(11),3,5,9-pentaene)-2,2,2-trifluoro-ethanone (100 g) in methanol (600 ml) was treated with an aqueous solution of sodium carbonate (69.05 g in 625 ml water). The mixture was warmed to 65° to 70° C. for 2 hours, and the reaction was monitored by HPLC/TLC (MDC:MeOH 9:1). The methanol was distilled out completely under vacuum. The residue was dissolved in water (1 l). The reaction mass was cooled to 25° to 30° C., and extracted with MDC (500 ml.×1). The combined organic layer was washed with DM water (1×500 ml). The organic layer was concentrated to obtain the product, i.e., Varenicline Base.

### Example 17

### Procedure for Preparation for 5,8,14-Triazatetracyclo-[10.3.1.0^{2,11}.0^{4,9}]hexadeca-2(11),3,5,7,9-pentaene (Varenicline Base)

A mixture of 1-(5,8,14-Triazatetracyclo[10.3.1.0^{2,11}.0^{4,9}]hexadeca-2(11),3,5,9-pentaene)-2,2,2-trifluoro-ethanone (200 g) and methanol (1.2 l) was treated with an aqueous solution of sodium carbonate (138 g in 1.2 l water) at 25° to 30° C. The mixture was warmed to 65° to 70° C., and stirred for 1.5 hours. The reaction was monitored by HPLC/TLC. Excess methanol was distilled out. The residue was dissolved in water (2 l) at 55° to 60° C., and stirred for 30 minutes to obtain a clear solution. The reaction mass was cooled to 25° to 30° C., and extracted with MDC (3.0 l×1). The combined organic layer was washed with DM water (1×1.0 l). The organic layer was concentrated to obtain the product, i.e., Varenicline Base (yield 125 g, HPLC purity 99.18 percent).

### Example 18

### In Situ Procedure for Preparation 1-(4,5-Dinitro-10-aza-tricyclo[6.3.1.0^{2,7}]dodeca-2(7),3,5-triene-10-yl)-2,2,2-trifluoro-ethanone (IX)

MDC (500 ml) 1,2,3,4-Tetrahydro-1,4-methano-napthalene-2,3-diol (50 g) and DM water (1300 ml) were charged into a 3.0 l, 4-neck round bottom flask equipped with a mechanical stirrer and Thermopocket. The resulting solution was cooled to 10° to 15° C., and sodium periodate (63.8 g) and benzyl triethyl ammonium chloride (0.65 g) were added at 100 to 15° C. The resulting mixture was stirred for 1 hour at 20° to 25° C. Progress of the reaction was monitored by TLC (ethyl acetate:hexane (5:5)). The layers were separated, and the aqueous layer was extracted with MDC (1×250 ml). The combined organic layer washed with DM water (2×250 ml), and the organic layer was dried over sodium sulfate (5 g) and collected in round bottom flask. To this was added benzyl amine (31.95 g), and the mixture was stirred for 2 minutes (solution-A).

Prior to the addition of benzyl amine to the dried organic layer to form solution-A, sodium triacetoxy borohydride (150.5 g) was charged in MDC (750 ml) in a second dry round bottom flask, and cooled to 0° to 5° C., and solution-A was added over a period of 5 to 10 minutes. The resulting mixture was allowed to warm to room temperature, and stirred for 1.5 hours at 200 to 25° C. The progress of the reaction was monitored by TLC (ethyl acetate:hexane (5:5)). The reaction was quenched by adding 10 percent aqueous sodium carbonate solution (1750 ml) to adjust the pH to 8 to 9. The resulting mixture was stirred for 1 hour at 20° to 25° C. The layers were separated, and the aqueous layer was extracted with MDC (1×250 ml). The combined organic layer was washed with 10 percent aqueous potassium dihydrogen phosphate (2×500 ml) and with DM water (250 ml) at 20° to 25° C. successively. Concentration afforded a oil product, which was dissolved in methanol (248 ml) and MeOH:HCl (52.96 gm-15-20% methanolic hydrochloric acid solution) in a 1 l autoclave at 25° to 30° C. To the above solution was added 20 percent palladium hydroxide on carbon (16.12 g). The resultant mixture was stirred under hydrogen at a pressure of 5.5 to 6.0 kg/cm² for 5 to 8 hours at 35° to 40° C. Progress of the reaction was monitored by HPLC until the starting material was present in an amount of no more than 1 percent. The reaction mass was filtered through a hyflo bed, and the resultant filtrate was concentrated to afford a solid product MDC (736 ml) was charged into the solid compound at 20° to 25° C. Triethyl amine (57.13 g) was added to this suspension at 0° to 5° C. To the solution was added tri-fluoro acetic anhydride (59.29 g) at 0° to 5° C. over a period of 30 to 40 minutes. The resulting mixture was allowed to warm to 25° to 30° C., and maintained with stirring for 2 hours. Progress of the reaction checked by TLC (ethyl acetate:hexane:ammonia (7:3:1 drop)). The resulting reaction mixture was quenched by the addition of DM water (345 ml) at 5° to 10° C., and stirred for 0.5 hour. The layers were separated, and the aqueous layer was extracted with MDC (1×184 ml). The combined organic layer was washed with 1N HCl (3×276 ml) and then 10 percent aqueous sodium chloride solution (1×184 ml), and set aside (solution A)

MDC (220.8 ml), tri-fluoro methane sulfonic acid (152.7 g) at 20° to 25° C. were charged in a second clean and dry 1.0 l 4-neck round bottom flask equipped with mechanical stirrer, thermopocket, and addition funnel. The solution was then cooled to 0° to 5° C., and charged with fuming nitric acid (34.1 g) over 25 to 35 minutes at 0° to 5° C. The resulting solution was stirred for 10 to 15 minutes at 0° to 5° C. The MDC layer, i.e., solution A, was added to the mixture over 1.0 to 1.5 hours at 0° to 5° C. The resulting reaction mixture was warmed to 250 to 30° C., and maintained for 2.5 hours. Progress of the reaction was checked by HPLC. The reaction was quenched by pouring the reaction mixture into DM water (552 ml) at 0° to 15° C., and maintained with stirring for 1.0 hour. The layers were separated, and the aqueous layer was extracted with MDC (2×331.2 ml). The combined organic layer was washed successively with DM water (3×331.2 ml), 8 percent aqueous NaHCO₃ solution (1×276 ml), and DM water (1×220.8 ml). The resulting organic layer was concentrated to obtain crude 1-(4,5-Dinitro-10-aza-tricyclo[6.3.1.02,7]dodeca-2(7),3,5-triene-10-yl)-2,2,2-trifluoro-ethanone. To the above crude product was added ethyl acetate (73.4 ml), and the resulting mixture was stirred for 2 hours at 55° to 60° C. The resultant mixture was cooled to 25° to 30° C., and stirred for 2 hours. The solid was filtered and washed with chilled ethyl acetate (73.4 ml) to obtain pure 1-(4,5-Dinitro-10-aza-tricyclo[6.3.1.02,7]dodeca-2(7),3,5-triene-10-yl)-2,2,2-trifluoro-ethanone (Yield-48.5 g HPLC purity—not less than 98.87%).

We claim:

1. A process for purifying a compound of formula (XI), 1-(5,8,14-Triazatetracyclo[10.3.1.0^{2,11}.0^{4,9}]hexadeca-2(11),3,5,9-pentaene)-2,2,2-trifluoro-ethanone, comprising contacting a compound of formula (XI) with an acid.

2. The process of claim 1, wherein the acid is an organic acid or mineral acid.

3. The process of claim 2, wherein the mineral acid is selected from the group consisting of hydrochloric acid, hydrobromic acid, hydroiodic acid, sulfuric acid, and mixtures thereof.

4. The process of claim 3, wherein the mineral acid is hydrochloric acid, sulfuric acid, or a mixture thereof.

5. The process of claim 2, wherein the organic acid is selected from the group consisting of lactic acid, acetic acid, formic acid, citric acid, oxalic acid, and mixtures thereof.

6. The process of claim 1, wherein the acid is hydrochloric acid.

7. The process of claim 1, wherein the acid is at a concentration of about 0.5N to about 2N.

8. The process of claim 7, wherein the acid is at a concentration of about 1.5N.

9. The process of claim 1, wherein the acid is added at a temperature of about 35° C. to about 45° C.

10. The process of claim 1, wherein addition of the acid results in a mixture having a pH of about 0.5 to about 1.

11. The process of claim 1, wherein the acid is aqueous.

12. The process of claim 1, wherein the compound of formula XI is recovered by concentration using reduced pressure, followed by filtration.

13. The process of claim 12, wherein concentration is performed at a temperature of about 35° C. to about 40° C.

14. The process of claim 1, wherein the purified compound of formula (XI) has a purity of about 98 percent to about 100 percent by area HPLC.

15. The process of claim 14, wherein the purified compound of formula (XI) has a purity of about 99 percent.

16. A process for purifying a compound of formula (VI), 10-Benzyl-10-aza-tricyclo[6.3.1.0^{2,7}]dodeca-2(7), 3,5-triene, comprising contacting the compound of formula VI with water and an alkali metal dihydrogen phosphate.

17. The process of claim 16, wherein the alkali metal dihydrogen phosphate is selected from the group consisting of potassium dihydrogen phosphate and sodium dihydrogen phosphate.

18. The process of claim 16, wherein the alkali metal dihydrogen phosphate is potassium dihydrogen phosphate.

19. The process of claim 16, wherein the potassium dihydrogen phosphate is used in about a 5 percent to about 25 percent aqueous solution.

20. The process of claim 19, wherein the potassium dihydrogen phosphate is used in about a 10 percent aqueous solution.

21. The process of claim 16, wherein potassium dihydrogen phosphate is added at a temperature of about 20° C. to about 30° C.

22. The process claim 16, wherein the water is demineralized water.

23. The process of claim 16, wherein the compound of formula (VI) is recovered by concentration using reduced pressure.

24. The process of claim 23, wherein concentration is performed at a temperature of about 35° C. to about 40° C.

25. The process of claim 16, wherein the purified compound of formula (VI) has a purity of about 95 percent to about 97 percent by area HPLC.

26. The process of claim 25, wherein the purified compound of formula (VI) has a purity of about 96.4 percent.

27. A process for preparing Varenicline base, comprising: a) Adding a di-halo substituted benzene in the presence of a solvent and a haloalkane to cyclopentadiene to obtain a compound of formula (IV), 1,4-Dihydro-1,4-methano-naphthalene in a Grignard reaction;
b) Treating the compound of formula (IV) obtained in step (a) with a catalyst in the presence of a solvent and, subsequently, an oxidizing agent to obtain a compound of formula (V), 1,2,3,4-tetrahydro-1,4-methano-naphthalene-2,3 diol;
c) Adding an oxidizing agent, a phase transfer catalyst, a protecting agent, and a reducing agent to the compound of formula (V) obtained in step (b) to obtain a compound of formula (VI), 10-benzyl-10-aza-tricyclo[6.3.1.0^{2,7}]dodeca-2(7),3,5-triene, and, optionally, purifying the compound of formula VI in a process step, comprising contacting the compound of formula VI with water and an alkali metal dihydrogen phosphate;
d) Adding HCl to the compound of formula VI obtained in step (c) to obtain a compound 10-benzyl-10-aza-tricyclo[6.3.1.0^{2,7}]dodeca-2(7), 3,5-triene HCl, and, subsequently, debenzylating the 10-benzyl-10-aza-tricyclo[6.3.1.0^{2,7}]dodeca-2(7),3,5-triene HCl to obtain a compound of formula (VII), 10-aza-tricyclo[6.3.1.0^{2,7}]dodeca-2(7)3,5-triene hydrochloride;
e) Adding a solvent and a fluorinating agent to the compound of formula (VII) obtained in step (d) to obtain a compound of formula (VIII), 1-(10-aza-tricyclo[6.3.1.0^{2,7}]dodeca-2(7),3,5-triene 10-yl)-2,2,2-trifluoro-ethanone;
f) Adding a nitrating source in the presence of a solvent and a Lewis acid to the compound of formula (VII) obtained in step (e), to obtain a compound of formula (IX), 1-(4,5-dinitro-10-aza-tricyclo[6.3.1.0^{2,7}]dodeca-2(7), 3,5-triene-10-yl)-2,2,2-trifluoroethanone, and, subsequently, reducing the compound of formula (IX) by hydrogenation to obtain a compound of formula (X), 1-(4,5-Diamino-10-aza-tricyclo[6.3.1.0^{2,7}]dodeca-2(7),3,5-triene-10-yl)-2,2,2-trifluoro-ethanone;
g) Cyclising the compound of formula (X) obtained in step (f) with a 40 percent aqueous glyoxal solution to obtain a compound of formula (XI), 1-(5,8,14-triazatetracyclo[10.3.1.0^{2,11}.0^{4,9}]hexadeca-2(11),3,5,9-pentaene)-2,2,2-trifluoro-ethanone; and purifying the compound of formula XI in a process step, comprising combining the compound of formula XI with an acid; and
h) Deprotecting the compound of formula XI obtained in step (g) to obtain Varenicline base.

28. The process of claim 27, wherein the fluorinating agent is trifluoroacetic anhydride.

29. The process of claim 27, wherein the di-halo substituted benzene in the presence of a solvent and a haloalkane is added to the cyclopentadiene under an inert atmosphere over a time period of about 2 to about 4 hours, and at a temperature in the range of about 50° to about 70° C.

30. The process of claim 27, wherein the compound of formula (IV) obtained in step (a) is treated with a catalyst in the presence of a solvent and, subsequently, an oxidizing agent, over a period of about 2 to about 8 hours, at a temperature of about 65° to about 70° C.

31. The process of claim 27, wherein the compound of formula VI is purified in the process step, comprising contacting the compound of formula VI with water and an alkali metal dihydrogen phosphate.

32. A process for preparing Varenicline L-tartrate comprising, obtaining Varenicline base according to process of claim 27, and converting the obtained Varenicline base to Varenicline L-tartrate.
